(19) United States
(12) Patent Application Publication
(10) Pub. No.: US 2006/0265033 A1
(43) Pub. Date: Nov. 23, 2006

(54) Tanning lamp

(76) Inventor: Candido Bruto, Milano (IT)

Correspondence Address: AKERMAN SENTERFITT, P.O. BOX 3188, WEST PALM BEACH, FL, 33402-3188, US

(21) Appl. No.: 10/551,307

(22) PCT Filed: Oct. 1, 2004
(86) PCT No.: PCT/EP04/10946

§ 371 Date: May 15, 2006

(30) Foreign Application Priority Data
Oct. 3, 2003 (IT) MI2003A 001904

(51) Int. Cl. A61N 5/06

(52) U.S. Cl. 607/94

(57) ABSTRACT

The present invention concerns a tanning lamp (1) for tanning skin, which is suitable for being installed in any rooms; the tanning lamp (1) comprises a panel (2), a UV radiation source (3) mounted on it and a 5 mobile shutter (4) associated with said panel (2).

### FIELD OF APPLICATION

The present invention refers, in general, to the technical field of the health item industry and, in particular, the invention concerns a lamp capable of emitting ultraviolet radiation or UV-A radiation, and therefore defined hereafter as tanning lamp.

### PRIOR ART

For some time tanning apparatuses comprising lamps capable of emitting ultraviolet radiation or UV-A radiation, used for tanning skin mostly in beauty centres, have been known.

The spread of such apparatuses, commonly defined as tanning lamps and generally made in the form of actual tanning stations like sun beds, booths or showers, has not reached homes yet, but to a minimum extent.

Such a limited spread is due both to the high cost of these tanning apparatuses possess, and to some other drawbacks from which they suffer, the main one of which is represented by the large space required to install them properly.

Furthermore, such apparatuses, although meeting the predetermined purpose, necessarily require, in addition to the aforementioned free space, rooms suitable for their appropriate positioning, like bathrooms, spare rooms or dressing rooms - rooms that rarely offer much space to allocate to housing such tanning lamps, with their big and bulky structure.

There are also smaller tanning lamps available on the market that are mainly intended for use at home.

Generally, lamps of this type comprise removable and modular structures made to be assembled at the time of use and then, at the end of the tanning treatment, disassembled and stowed away, for example, in a closet or cupboard.

Although advantageous under various points of view, this type of tanning lamp also is not without drawbacks, the main one of which consists of the assembly and disassembly operations that must be carried out each time the lamp is used.

Such operations, indeed, constitute an effort for the user and also involve a substantial loss of time, with the result of dissuading the user from using the tanning lamp.

### SUMMARY OF THE INVENTION

The technical problem underlying the present invention is that of devising a tanning lamp having structural and functional characteristics such as to overcome the aforementioned drawbacks, in other words such as to be structurally simple, thus allowing a low manufacturing cost and having a limited bulk with respect to that of tanning apparatuses available up to now, and also such as to be suitable for being positioned in any room, in particular at home.

The above object is accomplished by a tanning lamp of the type considered above, including a panel and a UV radiation source mounted on said panel and characterised in that it comprises a mobile shutter, or door, associated with said panel for removably, i.e. temporarily, blocking or masking said UV radiation source.

Further characteristics and advantages of the present invention shall become clearer from the description, made hereafter, of an example embodiment thereof, given for indicative and not limiting purposes, with reference to the attached drawings.

### BRIEF DESCRIPTION OF THE DRAWINGS

In such drawings:

FIG. 1 is a front view of a tanning lamp according to the present invention;

FIG. 2 is a top view of the tanning lamp of FIG. 1;

FIG. 3 is a perspective view during the operation of the tanning lamp of FIG. 1;

### DETAILED DESCRIPTION

With reference to the aforementioned figures, a tanning lamp made according to the present invention is globally indicated with 1. Said tanning lamp 1 essentially comprises a panel 2 and a UV radiation source mounted on it and generally indicated with 3.

In accordance with the present invention, the tanning lamp 1 is equipped with a mobile shutter or door 4, associated with said panel 2 to mask the source 3, the mobile shutter 4 being removable to carry out a temporary blocking of said UV radiation source 3, as shall become clearer in the rest of the description.

The tanning lamp 1, in accordance with a preferred embodiment, is equipped with a mirror-like reflecting wall 5 associated with the mobile shutter 4 at an outer surface 4a of the mobile shutter 4, with which it substantially constitutes a single piece.

Advantageously, the panel 2 is a box-type body 6, defining a space 6a, inside which the UV radiation source 3 is housed.

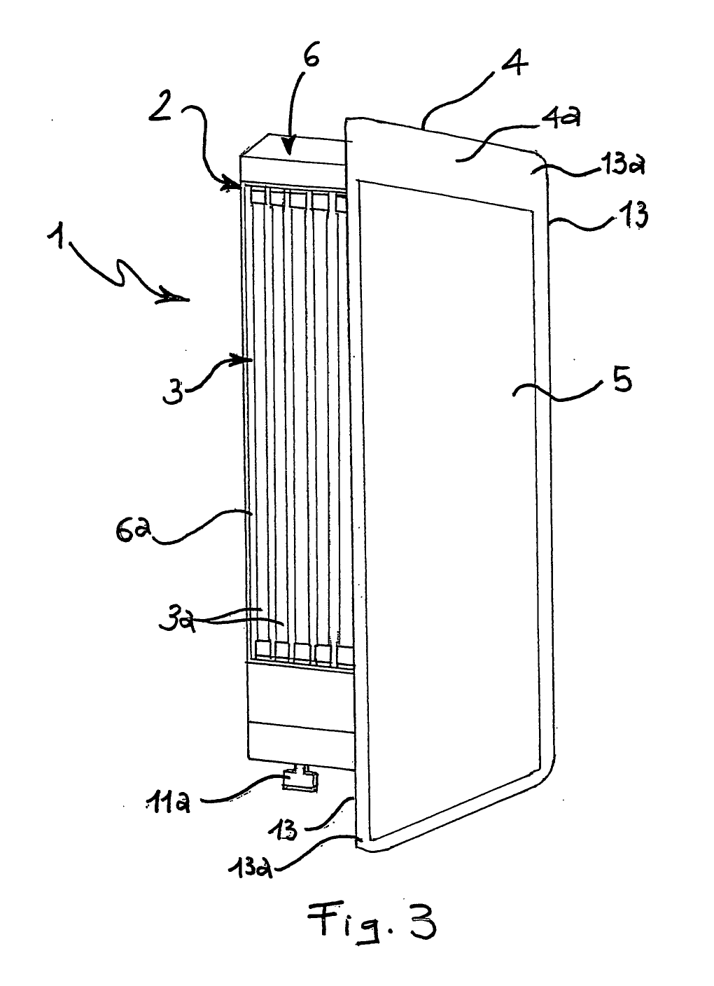

Preferably, such a UV radiation source 3 comprises a plurality of tubular ultraviolet light lamps 3a, mounted on respective connections 3b, and extending longitudinally inside the space 6a of the box-type body 6, as schematically illustrated in FIG. 3, which shows a perspective view of the tanning lamp 1.

The tubular lamps 3a can be mounted on a ceiling light, which is not illustrated since it is conventional.

The box-type body 6 is substantially shaped as a flat parallelepiped with a rectangular section, in which a rear wall 7, a first side wall 8 opposite a second side wall 9, a top wall 10 and a bottom wall 11 are defined.

The tanning lamp 1 is also equipped with means 12 for associating the mobile shutter 4 with the panel 2.

It should be noted that the associating means, according to a preferred embodiment, take the form of a plurality of book-type hinges 12, fixed at a first wing thereof to the mobile shutter 4, and at a second wing thereof, to the first side wall 8 of the box-type body 6.

Advantageously, the mobile shutter 4 has longitudinal edges 13 at which curved end portions 13a are defined, so as to define front rims having a rounded profile in the tanning lamp 1.

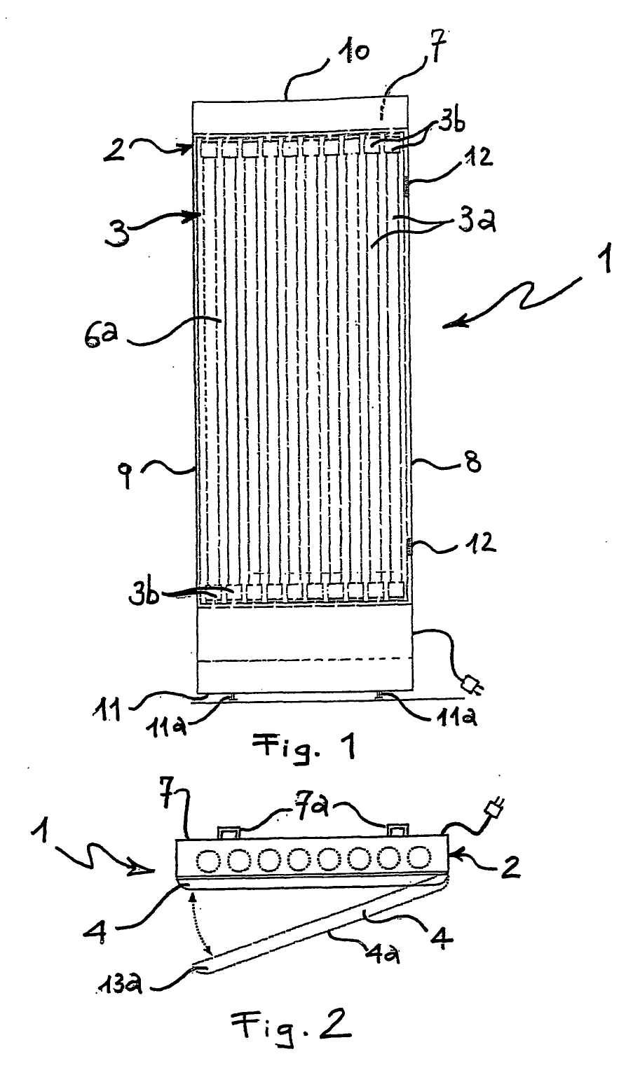

Advantageously, the tanning lamp 1 is equipped, at the bottom wall 11 of the box-type body 6, with a plurality of support feet 11a, in FIG. 1 two in number, which are height-adjustable; furthermore, it is equipped with fastening or attachment means 7a, in the form of hooks, associated with the rear wall 7 of the box-type body 6.

The tanning lamp 1 also has, at the top walls 10 and the bottom wall 11 of the box-type body 6, a plurality of slits or grids, which constitute openings for the dissipation of the heat developed during the operation of the lamps 3a, which are not represented since they are conventional.

To complete it, the tanning lamp 1 is equipped with electric circuits and electronic devices for controlling the lamps 3a, as well as an electric power supply cable and a switch for switching the lamps 3a on and off, not illustrated since they are per se known.

The switching on and off of the lamps 3a can also be carried out and programmed remotely by means of a remote control, whose signal is received by a corresponding sensor arranged on the panel 2.

Advantageously, both the box-type body 6 of the panel 2 and the mobile shutter 4 of the tanning lamp 1 are made from a light and strong material, for example aluminium.

In use, the tanning lamp according to the present invention is rested on the floor of the room where it is intended to be installed; after being put in a perfect vertical position, thanks to the adjustment of the support feet 11a foreseen on the bottom wall 11, which advantageously allow possible irregularities of the floor itself to be remedied, the lamp is attached or rather fixed to the wall, thanks to the fastening means 7a with which it is equipped at its rear wall 7.

The tanning lamp 1, once installed and positioned in the pre-selected room, thus looks like a fitting or like a piece of furniture, for example a cupboard, a wardrobe or a dressing table thanks to its particular structure, its flat shape and the mobile shutter 4, which masks, i.e. hides, the UV radiation source 3 from view.

If needed, it is possible to activate the tanning function of the lamp by simply switching on such a UV radiation source, and intervening, in other words by opening the mobile shutter 4, which in practice performs the function of a closing door or shutter for the space 6a of the box-type body 6, and therefore for the tanning lamp 1.

In this way the tanning lamp, in vertical position and fixed to the wall, minimises its bulk, at the same time always remaining available for quick and immediate use.

The main advantage of the present invention lies in the possibility of having a tanning lamp that, due to its structural characteristics, is suitable for being positioned in any room, thanks to the aforementioned reduced bulk thereof and above all thanks to the mobile shutter, which, masking the UV radiation source, makes the tanning lamp look like a common piece of furniture.

A further advantage of the tanning lamp according to the present invention lies in that it is possible to take care of its maintenance in a simple manner. Indeed, thanks to the mobile shutter, the space 6a of the box-type body 6 can easily be accessed for possible replacement of the tubular lamps 3a, or for other maintenance or repair interventions, as well as for cleaning, without having to detach the tanning lamp from the wall to which it is fixed.

It should also be said that the tanning lamp according to the present invention is well suited to be realized in an aesthetically agreeable manner so as to adorn the room for which it is intended, being able to be used not only as a tanning lamp, but also as a light source of great and beautiful aesthetic effect.

In such a case, one can foresee that the mobile shutter 4, in its closed position, in other words when it is masking the source 3, leaves a portion of the source 3 uncovered or at least visible to obtain such a light source.

Furthermore, the tanning lamp is structurally simple, so that it can be produced on a large and extremely large scale at low cost.

Obviously, the described tanning lamp can undergo numerous variants and modifications; for example, for masking the tubular lamps 3a, two doors 4 can be provided in the form of mobile half-shutters, the first one being associated with the first side wall 8 of the box-type body 6 and, respectively, the second one with the second side wall 9 of said box-type body 6; in this way, the bulk space of the mobile shutters 4 is further decreased when they are in open position, i.e. when the tanning lamp is used for its tanning function.

In a further variant embodiment the tanning lamp according to the present invention can be made to stand on a table, so as to be suitable for being positioned, for example, on a dressing table or on a small table.

In this case, the table-top tanning lamp shall be small in size with respect to the tanning lamp to be fixed to a wall, allowing for example just the skin on the face to be tanned. As an alternative to the fastening means for the wall 7a, the table-top tanning lamp can have conventional support means on a plane, like for example a pedestal, a stand, a shank or prop and similar support means for supporting it on a dressing table in vertical position or slightly reclined back, or in any case in a position such as to allow its tanning function to be exploited.

Furthermore, the associating means 12 can comprise a plurality of pivots, integral with the first side wall 8 of the box-type body 6 and intended to receive a respective plurality of straps fixed to the mobile shutter 4. In other words, the plurality of pivots and hinges provide suitable pivot means for the movement of the mobile shutter 4 relative to the panel 2 of the tanning lamp 1.

Moreover, the fastening means to the wall can comprise, in addition to the aforementioned hooks 7a, fastening means like brackets, screws, nails and similar means for fastening to the wall.

A person skilled in the art can add other variants and modifications of the tanning lamp according to the present invention to those quoted previously, in order to satisfy contingent and specific requirements, all of which are, however, contained within the scope of protection of the invention as defined by the appended claims.

1. Tanning lamp including a panel and a UV radiation source mounted on the panel wherein it comprises at least one mobile shutter associated with the panel for a temporary masking of the UV radiation source.

2. Tanning lamp according to claim 1, further comprising a mirror-like reflecting wail associated with the mobile shutter at an outer surface thereof, with which it substantially constitutes a single piece.

3. Tanning lamp according to claim 1, wherein the panel is a box-type body, defining a space, in which the UV radiation source is housed.

4. Tanning lamp according to claim 3, wherein the box-type body is shaped as a flat parallelepiped with a rectangular section comprising a rear wall, a first and respectively a second side wall, a top wall, and a bottom wall.

5. Tanning lamp according to claim 4, further comprising a plurality of support feet, that are height-adjustable, associated with the bottom wall of the box-type body.

6. Tanning lamp according to claim 4, further comprising fastening or attachment means associated with the rear wall of the box-type body.

7. Tanning lamp according to claim 1, wherein the mobile shutter has, at its longitudinal edges, curved end portions so as to define front rims having a rounded profile in the tanning lamp.

8. Tanning lamp according to claim 1, wherein the mobile shutter comprises two mobile half-shutters.

9. Tanning lamp according to claim 1, wherein at least a portion of the UV radiation source is left permanently uncovered by, or visible through, the mobile shutter.

10. Tanning lamp according to claim 1, further comprising table-top support means.

11. Tanning lamp including:
a panel;
a UV radiation source mounted on the panel;
at least one mobile shutter associated with the panel for a temporary masking of the UV radiation source;
wherein the panel is a box-type body, defining a space, in which the UV radiation source is housed, the box-type body being shaped as a flat parallelepiped with a rectangular section comprising a rear wall, a first and respectively a second side wall, a top wall, and a bottom wall, a plurality of support feet, that are height-adjustable, being associated with the bottom wall of the box-type body.

12. Tanning lamp according to claim 11, further comprising a mirror-like reflecting wall associated with the mobile shutter at an outer surface thereof, with which it substantially constitutes a single piece.

13. Tanning lamp according to claim 11, further comprising fastening or attachment means associated with the rear wall of the box-type body.

14. Tanning lamp according to claim 11, wherein the mobile shutter has, at its longitudinal edges, curved end portions so as to define front rims having a rounded profile in the tanning lamp.

15. Tanning lamp according to claim 11, wherein the mobile shutter comprises two mobile half shutters.

16. Tanning lamp according to claim 11, wherein at least a portion of the UV radiation source is left permanently uncovered by, or visible through, the mobile shutter.

17. Tanning lamp according to claim 11, further comprising table-top support means.

18. Tanning lamp including:
a panel;
a UV radiation source mounted on the panel;
at least one mobile shutter associated with the panel for a temporary masking of the UV radiation source,
wherein at least a portion of the UV radiation source is left permanently uncovered by, or visible through, the mobile shutter.

19. Tanning lamp according to claim 18, further comprising a mirror-like reflecting wall associated with the mobile shutter at an outer surface thereof, with which it substantially constitutes a single piece.

20. Tanning lamp according to claim 18, wherein the panel is a box-type body being shaped as a flat parallelepiped with a rectangular section comprising a rear wall, a first and respectively a second side wall, a top wall, and a bottom wall and defining a space, in which the UV radiation source is housed, plurality of support feet, that are height-adjustable, being associated with the bottom wall of the box-type body.
